(19) United States
(12) Patent Application Publication
(10) Pub. No.: US 2002/0077349 A1
(43) Pub. Date: Jun. 20, 2002

(54) Method of treating age-related vision impairment

(76) Inventor: Nathan D. Hamilton, Palo Alto, CA (US)

Correspondence Address: Skinner, Sutton, Watson & Rounds, 548 California Street, Reno, NV, 89509, US

(21) Appl. No.: 09/844,485

(22) Filed: Apr. 27, 2001

Related U.S. Application Data
(63) Continuation-in-part of application No. 09/706,207, filed on Nov. 2, 2000.
(60) Provisional application No. 60/223,167, filed on Aug. 7, 2000.
(60) Provisional application No. 60/163,352, filed on Nov. 3, 1999.

(51) Int. Cl. A61K 31/385; A01N 43/26

(52) U.S. Cl. 514/440

(57) ABSTRACT

Disclosed herein are methods to treat age-related vision losses. The method comprises administering a combination of a carnitine and an oxidant. Preferably the oxidant is thioctic acid. Preferably 0.12 grams to 3 grams of carnitine (particularly ALC) and 0.12 and 1.5 grams of R-α-lipoic acid are administered. Optionally, coenzyme Q and/or creatine also are administered. Preferably 10 mg to 500 mg/day of coenzyme Q10 and 1 to 30 grams/day of creatine are administered.

### RELATED APPLICATIONS

This application is a continuation-in-part of U.S. application Ser. No. 09/706,207, now pending, which claims the benefit of U.S. Provisional Application No. 60/223,167, filed Aug. 7, 2000, and U.S. Provisional Application No. 60/163,352, filed Nov. 3, 1999.

### TECHNICAL FIELD

This invention is related to the prevention and amelioration of vision impairment due to aging and other causes. More specifically, this invention is related to the administration of micronutrients, such as an antioxidant, a carnitine product, and optionally coenzyme Q and/or creatine to those at risk of age-related vision loss.

### BACKGROUND OF INVENTION

With age prominent changes occur in the brain and include a decrease in brain weight, gyral atrophy, ventricular dilation, and selective loss of neurons within different brain regions (Kemper, Neuroanatomical and neuropathological changes during aging and dementia. In: Martin A L, Knoefel J E (eds). GERIATRIC NEUROLOGY (2nd ed). Oxford University Press, New York City, pp. 3-67, 1994). The relevance of these changes to behavioral measurements is still largely ambiguous (e.g., Lezak, NEUROPSYCHOLOGICAL ASSESSMENT (3rd ed). Oxford University Press, New York, 1995). In addition to biological changes, environmental contexts are reflected in age-related cognitive changes (Arbuckle et al., Psychol Aging 7: 25-36, 1992). Recent studies with advanced brain imaging methods (especially PET and functional MRI) have elucidated the neuroanatomical localization of cognitive functions (e.g., Frackowiak, Trends Neurosci 17: 109-115, 1994; Moscovitch et al., Proc Natl Acad Sci USA 92: 3721-3725, 1995; Schacter et al., Proc Natl Acad Sci USA 93: 321-325, 1996). So far, very few of these studies have considered the effects of aging (Eustache et al., Neuropsychologia 33: 867-887, 1995; Grady et al., Science 269: 218-221, 1995). However, some associations between age-related cerebral and cognitive changes have been suggested.

Eustache et al. (1995, ibid.) demonstrated concomitant age-related declines in brain oxidative metabolism (in the resting condition) and tests of episodic memory, which also suggests that neurobiological changes within the neural network may underlie the vision impairments of normal aging. Accordingly, Grady et al. (1995, ibid.) found age-related reductions in regional cerebral blood flow within the network including the hippocampus and the anterior cingulate cortex during the encoding phase of a face recognition task.

By using a structural equation model, Jones et al. (Exp Aging Res 17: 227-242, 1991) found evidence for a causal relationship between age-related changes in the brain (as measured by CT and EEG) and function in healthy individuals.

Coenzyme Q or ubiquinone plays a central role in the mitochondrial respiratory chain that captures energy from metabolism. It exists in mitochondria in the oxidized quinone form under aerobic conditions. In the reduced form ubiquinol, Q10 is an antioxidant. Q also is present in mitochondrial lipids. The structure of Q is very similar to those of vitamins K and E, which are characterized by a polyisoprenoid side chain. Coenzyme Q10 has ten polyisoprenoid side chains. Mitochondria need to maintain a large excess of Q10, compared to other respiratory enzymes. Q10 is required to act on a mobile component of respiration that collects reducing equivalents from the more fixed complexes and passes them to other compounds.

Many conflicting reports have been published on the effectiveness of Q10 in various laboratory and clinical settings. Barbiroli et al reported that Q10 administration caused marked improvement in oxidative phosphorylation in both skeletal muscles and brains of patients with mitochondrial cytopathies due to enzyme defects (Biochimie 80(10): 847-53, 1998). On the other hand, Lass et al studied the Q10 and Q9 content in brain, heart, skeletal muscle and other organs but found a decrease in mitochondrial Q9 and Q10 only in aging skeletal muscle (Biofactors 9(2-4):199-205, 1999).

Life-long Q10 supplementation was studied in male rats and mice. Q10 did not prolong or shorten the lifespan of rats or mice. Plasma and liver levels were 2.6-8.4 times higher in the supplemented rats. Q10 levels in kidney, heart and brain were not affected by Q10 supplementation (Lonnrot K et al. Biochem Mol Biol Int 44(4):727-37, 1998).

To determine if Q10 has a neuroprotective effect, mice were first treated with Q10 or a control diet for four weeks. Then their striatal nerves were poisoned 1-Me-4-Ph-1,2,3,tetrahydropyridin (MPTP). The mice continued on their assigned diets for another week before sacrifice. Both groups had considerable brain damage; however, the Q10-treated mice had 37% higher dopamine and 62% more dense neurons, indicating a protective effect of Q10. (Beal MF et al. Brain Res 783(1):109-14, 1998.

Q10 also blocks the effects of doxorubicin, which by itself stimulates mitochondrial oxidant production and a marked increase in mtDNA deletions in cardiac tissue (Adachi et al. Biochem Biophys Res Commun 195:945-51, 1993).

A group of healthy Finnish men and women aged 28-77 were tested for the total peroxyl radical-trapping capacity of human plasma LDL phospholipids. There was an age-related difference in men, but not in women. Most of the decrease occurred before age 50, remaining low into the 70's. Supplementation with Q10 doubled the peroxyl radical-trapping capacity and thus may decrease LDL oxidation, which contributes to atherosclerosis (Aejmelaeus R et al. Mol Aspects Med 18(Supp):S113-20, 1997).

Creatine is present in muscular tissue and the heart. Small amounts are found in the blood but not in normal urine. Normally the liver and kidneys produce creatine. When creatine is metabolized, its end product is creatinine, which is excreted in the urine. Serum creatinine may increase with age. Muscle mass usually decreases with age, but it is unknown if it is entirely due to declining activity with age. Also, many older people do not eat as much meat, an important source of creatine. The greater part of creatine in muscle is combined with phosphoric acid as phosphocreatine. There it plays an important part in mitochondrial metabolism. In the mitochondria, creatine kinase isoenzymes transfer high-energy phosphate to creatine. Next, creatine phosphate is transported out of the mitochondria into the cell's environment where it generates extramitochondrial ATP. Different isoenzymes of creatine kinase mediate transfer of high-energy phosphate to and from the various systems that utilize or generate it, e.g., muscle contraction and glucose metabolism.

Researchers administered creatine and have studied a number of different parameters including aging and muscle function. Acute supplementation (5 days) in men over 60 was found to have no effect in isometric strength and only small increases in isokinetic performance and body mass (Rawson E S, Clarkson P M, Int. J. Sports Med 21(l):71-5, 2000). Another study reported results on older adults (67-80 years, 16 females, 16 males) who were randomly assigned to control-creatine, control-placebo, trained-creatine and trained-placebo groups for an 8-week test. Both groups of trained subjects had significant increases in 1- and 12-repetitions maxima, but no beneficial effect was observed for creatine supplementation (Bermon S et al. Acta Physiol Scand 164(2):147-55, 1998). On the other hand, when a different parameter directly related to muscle metabolism was measured, a positive effect was seen after 7 days. Groups of male and female 30-year-olds and 50-year-olds performed single-leg knee-extension exercises inside an MRI. At the start of the study, the older group had lower resting phosphocreatine (PCr) and lower mean initial PCr resynthesis rate. After creatine supplementation, the resting PCr increased 15% (P<0.05) in the young group and 30% (P<0.05) in the middle-aged group. In the middle-aged group, mean initial PCr resynthesis rate increased significantly (P<0.05), to a level comparable to that of the young group. The time to exhaustion was increased in both groups combined after creatine supplementation. Smith S A et al. concluded that creatine supplementation has a greater effect on PCr availability and resynthesis rate in middle-aged compared with younger persons (J Appl Physiol 85(4):1349-56, 1998).

Schuff N et al. analyzed age-related metabolite change and volume loss in the hippocampus by MRI (Neurobiol Aging 29(3):279-85, May-June 1999). They analyzed N-acetyl aspartate (NAA, a neuron marker), volume changes, and ratios of NAA/choline (Cho) and NAA/Cr (creatine). Volume decreased about 20% between 36 and 85 years, while NAA/Cho decreased by 24% and NAA/Cr decreased by 26%, all of which were significant. The Cho/Cr ratio remained stable. The volume loss correlated with neuronal marker loss and indicated loss of neurons. In contrast, Pfefferbaum A et al. (Magn Reson Med 41(2):276-84, 1999) reported NAA, Cho and Cr signal densities for healthy groups of 15 young and 19 elderly persons. NAA was higher in gray than white matter but did not differ between young and old subjects, despite significant gray matter volume deficits in the older subjects. The available gray matter appeared to be intact in older healthy adults. Cr concentrations were much higher in gray than white matter and significantly higher in the older subjects. Cho concentration in gray matter was also significantly higher in older subjects. The findings in older subjects were confirmed in another study in which Pfefferbaum compared Alzheimer disease (AD) and normal aging (Arch Gen Psychiatry 56(2):185-92, 1999). Both groups showed cortical gray matter volume deficits.

Nutritional deficiencies are known to contribute to poor neuron function. For example, the Wernicke-Korsakoff syndrome results from a failure to ingest thiamine. The patient has continued carbohydrate intake and gradually exhausts thiamine stores in critical areas of the thalamus and brainstem reticular formation. Underlying causes include dialysis, oversights in postoperative care, hyperemesis gravidarum and severe alcoholism. Memory for new information is severely affected but memory of distant events is less impaired. Therefore, the patient's previous experience is available to guide his actions and he may display little intellectual loss. Properly treated with fluids, calories and vitamin supplements, the condition dissipates over a period of weeks to months. Studies have demonstrated an association between the use of thiamin (vitamin B1), pyridoxine (vitamin B6) or cyanocobalamin (vitamin B12), and cognition. These vitamins are involved in carbohydrate metabolism.

Zinc levels are known to be low in the older population and zinc blood levels have positively correlated with psychological performance.

What is needed is improved nutrition to maintain brain function and prevent and ameliorate vision impairment.

### SUMMARY OF INVENTION

It is an object of the present invention to prevent and ameliorate the visual deficits which occur with aging. It is a further object to provide a combination of an effective amount of a suitable antioxidant and an effective amount of a carnitine to prevent and/or ameliorate the cognitive deficits associated with the above conditions.

A preferred combination of the present invention includes carnitine in the amount of 0.12 grams to 3 grams. A preferred form of carnitine is acetyl-L-carnitine (ALC).

A preferred combination of the present invention includes the antioxidant as R-α-lipoic acid in the amount of about 0.12 grams to about 1.5 grams.

Optionally, coenzyme Q and/or creatine also are administered. Preferably 10 mg to 500 mg/day of coenzyme Q10 and 1 to 30 grams/day of creatine are administered.

### DETAILED DESCRIPTION

Testing with PET, particularly enhanced with F-18 fluorodeoxyglucose (FDG) to analyze metabolic activity, has shown that two derived factors separated healthy persons below age 42 from those above age 48. Both secondary memory for material verbally processed in combination with Broca's metabolic ratio and tests requiring sequential or organizational coding of information (executive function) combined with metabolic measures of thalamic regions were greater for the younger group than for the older group. The investigators concluded that a frontal-subcortical decrement in metabolism is present in age-dependent memory processing. Riege W H et al. Brain Cogn 5(4): 412-27, 1986. Thus, it may be beneficial in the late 30's to early 40's to begin therapy which enhances metabolism and has been proven to counteract aging in mitochondria. These factors certainly could have an effect on age-related vision losses.

Carnitine and lipoic acid, and optionally coenzyme Q and/or creatine, are administered to discourage age-related vision loss and provide improved vision in older individuals and others with unhealthy mitochondria. Recent research has shown precisely how these compounds work to promote healthy mitochondria, which are the energy powerhouses of the cells. Mitochondria are responsible for the production of ATP and are present in relatively high numbers in essentially all cells of the body. The mitochondrial electron transport system consumes approximately 85% of the oxygen utilized by a cell. Cellular energy deficits caused by declines in mitochondrial function can impair normal cellular activities and compromise the cell's ability to adapt to various physiological stresses, a major factor in aging. Because of this high oxygen use, the mitochondria also have the highest production of oxidants.

Oxidants damage mitochondria in three important ways. Oxidants damage DNA, lipids and protein. The intra-mitochondrial DNA (mtDNA) have levels of oxidative damage which are at least 10-fold higher than those of nuclear DNA, which correlates with the 17-fold higher evolutionary mutation rate in mtDNA compared with nuclear DNA. mtDNA oxidation accumulates as a function of age, which has been shown in several species, including humans. This may lead to dysfunctional mitochondria. Mitochondrial protein damage is also age-related and may decrease energy production and increase oxidant production. Oxidative damage to mitochondrial lipids contributes to the decreasing fluidity of cell membranes with age. The lipid cardiolipin is a major component of the mitochondrial membrane and facilitates the activities of critical mitochondrial inner membrane enzymes. The aged, damaged mitochondrial membrane cannot contain the oxidants nor can it maintain as high a polarity as the younger membrane.

Fatty acid oxidation is an important energy source for many tissues. The activity of carnitine-acetyl-carnitine exchange across the inner mitochondrial membrane is of great importance. The activity of this exchange reaction is decreased significantly with age, which may be due to a lower intra-mitochondrial pool of carnitine. L-carnitine or ALC has been shown to slow or reverse this age-related dysfunction. It also can reverse the age-related decrease in cardiolipin, age-associated decrease in mtDNA transcription, and decreased membrane potential. By itself, L-carnitine or ALC cannot correct the problem of excess oxidants. In fact, it was recently reported that carnitine supplementation increased oxidant production by 30% and decreased cell antioxidants markedly. Thus, ALC administration alone in older individuals may contribute to greater oxidative stress.

For the age-compromised mitochondrial engines to run on all cylinders, both carnitine and lipoic acid are essential. Lipoic acid is an antioxidant. And R-α-lipoic acid is a mitochondrial enzyme, which can help reverse the decline in metabolism seen with age. R-α-lipoic acid supplementation has been shown to 1) reverse the age-related decrease in oxygen consumption, 2) restore the age-related decline in mitochondrial membrane potential, 3) triple the ambulatory activity of aged rats, 4) significantly lower the age-related increase in oxidants, and 5) restore glutathione and ascorbic acid levels to youthful levels.

Clearly, both carnitine and lipoic acid contribute to restoration of age-related mitochondria function and metabolic activity in individuals in which those were compromised. This contributes to improvements in energy, general health, mental acuity, immune system function, skin and hair appearance and muscle mass.

Carnitine is available in many forms and all those are included in the invention of the combination of carnitine and thioctic acid. Carnitine and carnitine derivatives have been used as metabolites in animal husbandry and for human diet and therapy. U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine, for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); U.S. Pat. No. 5,240,961 (Method of treating reduced insulin-like growth factor and bone loss associated with aging); etc. Most preferably, the carnitine is acetyl-L-carnitine.

A daily dosage of carnitine is about 10 mg to 8 g. Preferably the daily dose of carnitine is 25-1,000 mg. More preferably, the daily dose of carnitine is about 40-700 mg. Most preferably, the daily dose of carnitine is at least about 50 milligrams (0.05 g) per day.

By lipoic acid or thioctic acid is meant a mitochondrially active antioxidant which physiologically comprises a metabolically reactive thiol group. Mitochondrially active antioxidants including vitamins (especially C, E, B and D), glutathione, N-acetyl cysteine (NAC), lipoic acid, their derivatives, etc., have been used variously as human nutritional supplements and in dietary prophylaxis and therapy. For example, applications of lipoic acid have included U.S. Pat. No. 5,607,980 (Topical compositions having improved skin); U.S. Pat. No. 5,472,698 (Composition for enhancing lipid production in skin); U.S. Pat. No. 5,292,538 (Improved sustained energy and anabolic composition and method of making); U.S. Pat. No. 5,536,645 (Nutritive medium for the culture of microorganisms); U.S. Pat. No. 5,326,699 (Serum-free medium for culturing animal cells); etc. Preferably, the compound is at least one of glutathione, N-acetyl cysteine and lipoic acid. Most preferably, the compound is the Renantiomeric form of lipoic acid. Metabolites of lipoic acid have been found to have a longer half life and also are suitable for supplementation.

A daily dosage of lipoic acid is about 10 mg to 8 g. Preferably the daily dose of lipoic acid is 25-1,000 mg. More preferably, the daily dose of lipoic acid is about 40-700 mg. Most preferably, the daily dose of lipoic acid is at least about 50 milligrams (0.05 g) per day.

Q10 supplementation also is important. In groups of males and females ranging from 90-106 years, inadequate Q10 status was present in 40% for women and 24% for men. In women, the decreased Q10 was associated with impaired natural killer cell effectiveness (p<0.05), indicating decreased ability to fight infections and to quickly eliminate individual cancer cells as they first develop. Q10 also appears to block programmed cell death, or apoptosis, through its action in the mitochondria (Kagan T et al, Ann NY Acad Sci 887:31-47, 1999). Furthermore, Q10 in its reduced form of ubiquinol-10, which is normally present in the blood, appears to protect human lymphocytes from oxidative damage to DNA (Tomasetti et al, Free Radic Biol Med 27 (9-10):1027-32, November 1999). No important adverse effects have been reported from experiments using daily supplements of up to 200 mg Q10 for 6-12 months and 100 mg daily for up to 6 y. Overvad K et al. Eur J Clin Nutr 53(10):764-70, 1999.

Q10 also may contribute to the anti-aging effect by protecting against atherosclerosis which also results from oxidative stress. Pedersen H S, et al. Biofactors 9(2-4): 319-23, 1999). Protecting brain blood vessels will also help support brain function.

As for the appropriate dose of Q10, older Finnish men obtained benefit from 100 mg/day. A woman deficient in Q10 received 150 mg/kg and rapidly improved (Sobriera et al. Neurology 48:1283-43, 1997). Q10 has also been used at chronic doses of about 200 mg/day to improve heart function in persons with hypertrophic cardiomyopathy. Based on this information, a supplemental dosage ranges from about 10 mg/day to about 500 mg/day. Preferably, the Q10 dose is about 100 mg/day.

Because creatine is often deficient in older individuals, creatine supplementation is important. Many athletes have taken doses of creatine up to 75 grams a day for years without known adverse effects, aside from weight gain attributed to increased muscle mass. Creatine may be most beneficial when ingested with glucose, which tends to increase creatine absorption. Often athletes ingest loading doses of 20 g/day divided into four doses for 5 days to one week. Then they take a maintenance dose of 5 g/day. Benefit in one week in older individuals (40-73) has also been seen from a 20 g/day dose, in the form of increased skeletal muscle strength and endurance. It has been reported that 1.5g -25 g/day are safe for period of at least a year. A suitable dosage range is 0.5 g/day to 25 g/day, preferably 1-10 grams per day and most preferably about 5 g/day. Creatine is available as a salt, monohydrate, phosphate and citrate.

In addition to the compositions mentioned above and the examples given below, breakfast products would also benefit from the addition of a carnitine, a form of thioctic acid, and optionally Q10 and/or creatine. Examples of such breakfast products include, but are not limited to, breakfast cereal (Total®, etc.), breakfast bars, Poptart® pastry, and quick breakfasts in a bun or taco (e.g., McDonald® Egg McMuffin®). The carnitine, thioctic acid, and optionally Q10 and/or creatine can be added to bulk powders or powder packets, for example, in the following compositions: orange juice (e.g., Tang®), coffee creamer (e.g., Cremora®), powdered milk, powdered milk shakes/smoothies (e.g., MetaRX), butter-flavored powder, sweetener powders (e.g., Nutrasweet®), and spice and herb mixes. The combination of carnitine, thioctic acid, and optionally Q10 and/or creatine can be mixed with any cooked or uncooked food.

Premade drinks which would benefit from the inclusion of carnitine, thioctic acid, and optionally Q10 and/or creatine include, but are not limited to, pre-made smoothies, additives to drinks like Jamba Juice® and Starbucks®, sports drinks such as Gatorade®, diet drinks such as Weight Watchers® and Slim Fast®, and herbal drinks such as SoBe® (with St. John's Wort and other popular herbs). The formulations with carnitine, thioctic acid, and optionally Q10 and/or creatine also can include any fortified foods or meals replacement foods.

The combination of carnitine, thioctic acid, and optionally Q10 and/or creatine is provided in pet formulations, dried or canned or as a supplement for addition thereto. Animals expected to benefit from the composition include but are not limited to dogs, cats, horses, birds and fish.

The formulations and/or content of these products are on the product label or are otherwise publicly available.

Additional nutrients are particularly important in older individuals, including calcium, vitamins B12, B6, C, D or E, folic acid, niacin, iron and zinc. Many of these nutrients have been found to be deficient in the diets of elders and should be appropriately supplemented in nutritional beverages and bars.

A preferred formulation provides lipoic acid and carnitine, optionally in combination with coenzyme Q10 and/or creatine, in a timed release formulation to provide a steady supply of the nutrients to the mitochondria which work 24 hours a day. One method of accomplishing timed release is chemically combining the micronutrient(s) with other molecules, which generally slows the process of making the micronutrient(s) available. Also the use of different salts of the micronutrients with different dissolution rates provides for gradual and appropriate release of the product.

Besides these methods, two other basic systems are used to control release for oral administration: coating a core comprising the micronutrient(s) and excipients (coated system) and incorporating the micronutrient(s) into a matrix (matrix system). Coated systems involve the preparation of product-loaded cores and coating the cores with release rate-retarding materials. Product-loaded cores can be formulated as microspheres, granules, pellets or core tablets. There are many known core preparation methods, including, but not limited to, 1) producing granules by top spray fluidized bed granulation, or by solution/suspension/powdering layering by Wurster coating, 2) producing spherical granules or pellets by extrusion-spheronization, rotary processing, and melt pelletization; 3) producing core tablets by compression and coating with a release rate-retarding material; 4) producing microspheres by emulsification and spray-drying.

Matrix systems embed the micronutrient in a slowly disintegrating or non-disintegrating matrix. Rate of release is controlled by the erosion of the matrix and/or by the diffusion of the micronutrient(s) through the matrix. In general, the active product substance, excipients and the release rate-retarding materials are mixed and then processed into matrix pellets or tablets. Matrix pellets can be formed by granulation, spheronization using cellulosic materials, or by melt pelletization using release retardant materials, while matrix tablets are prepared by compression in a tablet press. An example of a cellulosic material is hydroxypropylmethylcellulose as the release rate-retarding material.

Coated or matrix pellets can be filled into capsules or compression tabletted. The rate of release can be further modified by blending coated or matrix pellets with different release rates of the same product to obtain the desired product release profile. Pellets containing any of lipoic acid, carnitine, coenzyme Q10 or creatine can be blended to form a combination product.

Convenient assays for the requisite bioactivities are described above or in the references cited herein. For example, cardiolipin content is readily assayed as referenced in Guan, Z. Z., Soderberg, M., Sindelar, P., and Edlund, C. Content and Fatty Acid Composition of Cardiolipin in the Brain of Patients with Alzheimer's Disease. Neurochem. Int. 25: 295-300, 1994 and oxidant production (DCFH) may be assayed as described by LeBel, C. P., Ischiropoulos, H., and Bondy, S. C. Evaluation of the Probe 2′,7′-Dichlorofluorescein as an Indicator of Reactive Oxygen Species Formation and Oxidative Stress. Chem. Res. Toxicol. 5: 227-231, 1992. Testing for vision deficits is well known in the art. PET scans and functional MRI can localize and quantitate the neuroanatomical localization of losses, too.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A method of treating age-related vision loss, the method comprising administering effective amounts of a suitable antioxidant, a carnitine and optionally coenzyme Q and/or creatine.

2. The method of claim 1 wherein the carnitine administered is ALC and the effective amount is in the range of 0.5 grams to 3 grams.

3. The method of claim 1 wherein the antioxidant administered is R-α-lipoic acid.

4. The method of claim 1 wherein the antioxidant is administered in the amount of 0.25 grams to 1.5 grams.

5. The method of claim 1 wherein the coenzyme Q is coenzyme Q10 and is administered in the amount of 10 to 500 mg/day.

6. The method of claim 1 wherein the creatine is administered in the amount of 1 to 30 grams/day.
